(19) United States
(12) Patent Application Publication
(10) Pub. No.: US 2007/0009448 A1
(43) Pub. Date: Jan. 11, 2007

(54) Silanol based dental treatment

(76) Inventor: John A. Kanca III, Middlebury, CT (US)

Correspondence Address: DORSEY & WHITNEY LLP;INTELLECTUAL PROPERTY DEPARTMENT, SUITE 1500, 50 SOUTH SIXTH STREET, MINNEAPOLIS, MN, 55402-1498, US

(21) Appl. No.: 11/360,979

(22) Filed: Feb. 23, 2006

Related U.S. Application Data
(60) Provisional application No. 60/656,187, filed on Feb. 25, 2005.

(51) Int. Cl. A61K 8/89

(52) U.S. Cl. 424/49

(57) ABSTRACT

Etching compositions, curable compositions, packaged products and methods of use for the treatment of bone substrate, i.e., teeth, are described. The etch solutions generally include an aqueous acidic solution with an ethylenically unsaturated monomer and a silane. The methods of the invention provide the ability to modify a bone or bone-like surface so that the curable composition, such as an adhesive resin, can be used in combination with a restorative material.

### CROSS REFERENCE TO RELATED APPLICATIONS

This application claims benefit of U.S. Provisional Patent Application No. 60/656,187, filed on Feb. 25, 2005, which is incorporated by reference in its entirety.

### FIELD OF THE INVENTION

The present invention relates to dental treatment (etch) solutions, reactive monomeric based adhesive compositions, packaged formulations that contain the etch solution and adhesive composition and methods for their use. More specifically, the present invention relates to a superior two step process, whereby an etch solution is applied to a substrate, such as a crown or a tooth and subsequently causes the substrate surface to become receptive to adhesive compositions. After the reaction has occurred, the adhesive composition is applied to the treated surface and the surface is readied for a restorative material.

### BACKGROUND OF THE INVENTION

Silane or silanol priming solutions have been used for many years for priming, etching and repair of dental materials such as porcelain, metals, alloys and dental composites. These solutions typically are sold as two-part products which are mixed together prior to use, or as one-part products which are used as is.

Representative two-part products include a first solution of water:isopropanol with a pH of about 4.5, and a second solution of about 20% gamma-methacryloxypropyltrimethoxysilane dissolved in isopropanol. Portions of each are mixed together and allowed to stand for a period of time prior to use, forming a solution containing water:isopropanol:silanol.

Representative one-part products include gamma-methacryloxypropyltrimethoxysilane and gamma-glycidoxypropyltrimethoxysilane in n-butanol. The one-part products contain essentially completely unhydrolyzed silane when in solution. Each is applied to an acid-treated substrate, such as a tooth or porcelain surface, and allowed to stand while the silane hydrolyzes in situ due to the action of acid and moisture.

Prior to the application of silanes or silanols to ceramic, bot not tooth surfaces, it is often recommended that the surface be treated with a buffered hydrofluoric acid, in concentrations ranging from 4-9% and for periods of 1 to 9 minutes. Hydrofluoric acid can etch the dental surface, leaving a microporous surface that is retentive for resin materials. However, there are always safety concerns when strong acids, such as hydrofluoric acid, are used in the oral cavity.

Hydrofluoric acid is ineffective for etching enamel and dentin, as it rapidly forms calcium fluoride and prevents further etching from occurring. An additional drawback associated with hydrofluoric acid on enamel or dentin is that the calcium fluoride that forms, prevents bonding to the surface with a resin and restorative material

Therefore, a need exists for new compositions, solutions and methods which overcome one or more of the disadvantages of currently available products.

### BRIEF SUMMARY OF THE INVENTION

The present invention provides a unique unexpectedly simple and easy to use bone, i.e., tooth, enamel, dentin, or porcelain etch solution, a curable adhesive bonding composition, packaged products containing either or both of the compositions, and methods to use the compositions of the invention. The systems of the present invention can be self-cured or can be treated with light energy to facilitate curing.

The present invention provides distinct advantages over presently known surface etching/bonding systems. Present etch solutions that contain hydrofluoric acid are required to be rinsed off porcelain surfaces prior to further treatment, e.g., with a resin. The hydrofluoric acid is also considered to be a catalyst to help promote the hydrolysis of the silane that is applied to the surface. In contrast to current silane/silanol compositions currently available for use in surface treatments, the present etching solution is not required to be rinsed off, removed and/or dried prior to the application of a bonding composition. Alternatively, currently available bonding systems include both an etch solution and the bonding resin as a one component application. Often times, the one component application systems do not bond well to the bone substrate because the etch solution interferes with the ability of the bonding resin to adhere to the substrate.

In one embodiment, the present invention provides a solution capable of etching a surface, such as a dental material. In one aspect the substrate is bone, dentin, enamel, gum, amalgam, metal, porcelain or plastic. The etching solution includes an aqueous acidic solution that includes a silane and an ethylenically unsaturated monomer. The pH of the solution is below about 4. Typically the components that make up the acidic solution include an organic and an inorganic acid, such as succinic acid and nitric acid. The ethylenically unsaturated monomer is generally an acrylate or a methacrylate such as acrylic acid or methacrylic acid and the silane is a trialkoxyl silane having a reactive moiety suitable for radical polymerization, such as gamma-methacryloxypropyltrimethoxysilane or gamma-glycidoxypropyltrimethoxysilane.

Not to be limited by theory, it is believed that the etch solution works well when the silane is at least partially hydrolyzed to a silanol. In one particular aspect, the solutions work well when the silane is completely hydrolyzed to a silanol. The hydrolysis or miscibility of the silane in the solution can be determined visually. When the aqueous component and silane are mixed together, hydrolysis of the silane occurs. This can be determined visually when at first a two phase solution/emulsion is formed as the silane does not appreciably dissolve or hydrolyze immediately. Over a short period of time, generally within a few seconds to a few minutes, the silane becomes miscible and or hydrolyzed with the aqueous solution so that a homogenous visually clear solution is prepared. It is not known if the silane is partially or fully hydrolyzed at this point and/or if it becomes miscible in the aqueous solution. In any event, a solution is prepared and can be used to treat various surfaces described throughout this specification.

Generally, the silane (or after partial or complete hydrolysis to a silanol) in the solutions of the invention is present in an amount between about 20 and about 60 percent by weight of the total weight of the solution. It is to be understood that the term “silane” as used herein, is intended to include both unhydrolyzed, partially hydrolyzed and fully hydrolyzed silanes. This is because it is difficult to determine when the silane is completely hydrolyzed to a silanol having no remaining hydrolyzable groups. The combination of silane, partially hydrolyzed silane and fully hydrolyzed silane to silanols may help to explain the unexpected ability of the present invention to help promote good adhesion between two surfaces and a bonding agent.

Suitable additional components in the etching solution include, for example, nitric acid and succinic or citric acid, or combinations thereof, organic solvents such as acetone or lower weight alcohols, and/or various surfactants.

The present invention also pertains to the use of curable composites in combination with the etch solutions of the present invention. The curable composites include (a) at least one ethylenically unsaturated functional monomer, (b) at least one polyethylenically unsaturated functional crosslinking monomer, (c) a (optionally) fluoride ion agent and/or optionally (d) a solvent and/or optionally water. More specifically, the ethylenically unsaturated monomer(s) of (a) is present in an amount of from about 0.5 to about 25 parts by weight. The polyethylenically unsaturated crosslinking monomer(s) of (b) is present in an amount from about 5 to about 40 parts by weight. The optional fluoride agent is present in an amount from about 0.5 to about 5 parts by weight, with the solvent and/or water being present in an amount to equal a total of 100 parts by weight of all components.

Suitable curable resin compositions include those that are commercially available such as OneStep (Bisco), All Bond 2 (Bisco), Prime & Bond, PB2, PBNT, Xeno 3 and 4 (LD Caulk), Tenure (DenMat), Excit (Ivoclar), AdheSE (Ivoclar), Optibond, Optibond FL, Optibond Solo, Optibond Solo Plus (Kerr), Scotchbond, Scotchbond 2, Single Bond, Scotchbond Multi-Purpose, Adper Prompt-L-Pop, Adper Single Bond Plus (3M), Gluma Comfort Bond, i-bond (Kulzer), PQ1, PermaQuik, PQ Clear (Ultradent), Clearfil SE Bond, LinerBond, LinerBond 2 (Kuraray), One-Up Bond (Tokuyama), Integrabond (Premier), SnapBond (Cooley), Cabrio (Discus), Easy Bond, Touch & Bond, Brush & Bond, AmalgamBond (Parkell), Prelude (Danville) and those described in pending U.S. Ser. No. 10/323,865, filed Dec. 18, 2002, entitled “Device for Dental Applications”, the contents of which are incorporated herein in their entirety for all purposes.

The curable composition generally includes a photoinitiator system. The photoinitiator system includes a light-sensitive initiator and a polymerization accelerator. A suitable light-sensitive initiator is camphorquinone (CQ) and a suitable polymerization accelerator is ethyl N,N-dimethyl-4-aminobenzoic acid or ethyldimethylaminobenzoic acid (EDMAB).

The present invention further provides packaged formulations of the etch solutions, the curable compositions and combinations thereof.

The present invention also provides method to use the etch solutions, alone or in combination with the curable compositions of the invention. It should be understood that the etch solutions of the present invention can be used with those commercially available bonding resins. The method of the invention involves applying an effective amount of an etching solution to the substrate. The etch solution can be any of the solutions described herein. The excess etching solution can be removed such that the substrate remains moist. This can be easily accomplished by touching an absorbent material to the surface and blotting the excess from the surface. Alternatively, the surface can be dried. Thereafter, a curable composite is applied to the moist substrate, i.e., a tooth.

While multiple embodiments are disclosed, still other embodiments of the present invention will become apparent to those skilled in the art from the following detailed description. As will be apparent, the invention is capable of modifications in various obvious aspects, all without departing from the spirit and scope of the present invention. Accordingly, the detailed descriptions are to be regarded as illustrative in nature and not restrictive.

### DETAILED DESCRIPTION

The present invention provides a unique unexpectedly simple and easy to use etch solution that can be used in combination with a curable adhesive bonding composition, packaged products containing the etch solution and/or the bonding composition, and methods to use the compositions of the invention. The present invention provides distinct advantages over presently known etching/bonding systems as described throughout the specification.

In one embodiment, the present invention provides a solution capable of etching a substrate surface, such as a dental material. In one aspect the substrate is bone, dentin, enamel, gum, amalgam, metal, porcelain or plastic. The etching solution includes an aqueous acidic solution that includes a silane and an ethylenically unsaturated monomer. The pH of the solution is below about 4.

It should be understood that throughout the specification, the etching solution can be utilized on any bone or bone-like substrate that includes, but is not limited to, apatite and hydroxyapatite. Bone is a complex mineralizing system composed of an inorganic or mineral phase, an organic matrix phase, and water. The inorganic mineral phase is composed mainly of crystalline calcium phosphate salts while the organic matrix phase consists mostly of collagen and other noncollagenous proteins.

The phrases “etching solution” or “etch solution” are recognized in the art and are intended to include the compositions of the invention that superficially dissolve or modify bone, bone-like substrates and dental materials. For example, in the case of teeth, the etch solution removes the smear layer and facilitates demineralization of the surface of the dental tissue.

The term “dental material” is intended to include dental porcelain, dental alloys, and cured dental composites, restoratives, zinc oxyphosphate, carboxylate cements, glass ionomer cements, compomers, resin-modified glass ionomers and adhesives, such materials being suitable for use in the oral environment.

As described above, the etch solutions of the present invention include several components. One of which is an acid to reduce the pH of the aqueous solution. The acid can be inorganic, organic or combinations of both.

Suitable inorganic acids include nitric acid, sulfuric acid, hydrochloric acid, phosphoric acid and mixtures thereof. In one aspect, the inorganic acid is nitric acid. Nitric acid is generally available as a 70 percent aqueous solution and is utilized at this concentration throughout this application. Sulfuric acid, hydrochloric acid and phosphoric acid are also available as 95-98 percent, 37 percent and 85 percent aqueous solutions, respectively, and are utilized at these concentrations throughout the application. Where noted as parts by weight, this refers to using the concentrated inorganic acid, at the respective commercial concentration, on a weight basis.

It should be noted that commercially available etch solutions that contain phosphoric acid do not work well in dental applications if the solution is not first rinsed from the substrate to which it is applied. This is due to the precipitation of calcium phosphate from the etch solution that is generated by the reaction of phosphoric acid with the calcium of the bone substrate. The present invention avoids such precipitation by either utilizing a different inorganic acid or by using an organic solvent, surfactant and/or organic acid which inhibit the deposition of calcium phosphate onto the cleaned bone substrate surface.

The inorganic acid can be present in the etch solution in an amount of from about 1 to about 10 parts by weight. Suitable ranges of inorganic acid include from about 2 to about 9 parts by weight, about 4 to about 8 parts by weight, and about 5 to about 7 parts by weight, i.e. about 6 to about 8 parts by weight, i.e., 5 parts by weight based on the total weight of the solution. In one embodiment, the inorganic acid is nitric acid.

The organic acid can be present in the etch solution in an amount of from about 1 to about 10 parts by weight. Suitable ranges of organic acid include from about 2 to about 9 parts by weight, about 4 to about 8 parts by weight, and about 5 to about 7 parts by weight, i.e. about 6 to about 8 parts by weight, i.e., 2.5 parts by weight based on the total weight of the solution.

Suitable organic acids include lactic acid, pyruvic acid, glycolic acid, acetic acid, chloroacetic acid, dichloroacetic acid, trichloroacetic acid, cyanoacetic acid, tartaric acid, succinic acid, glutaric acid, maleic acid, fumaric acid, malonic acid, citraconic acid, ortho-phthalic acid, meta-phthalic acid, para-phthalic acid, citric acid, tricarballyic acid, 1,3,5-pentanetricarboxylic acid and trimellitic acid and mixtures thereof. Other suitable organic acids include 2-acrylamido-2-methylpropane sulfonic acid, benzenesulfonic acid, benzoic acid, bromoacetic acid, 10-camphorquinonesulfonic acid, 10-camphorsulfonic acid, dibromoacetic acid, 2,4-dinitrophenol, formic acid, fumaric acid, 2-hydroxy-4-methoxybenzophenone-5-sulfonic acid, maleic-acid, 2-naphthalene sulfonic acid, nitric acid, oxalic acid, p-nitrophenol, phenol, phosphorous acid esters (such as 2,2′-bis(a-methacryloxy-b-hydroxypropoxyphenyl)propane diphosphonate (Bis-GMA diphosphonate), dibutyl phosphite, di-2-ethyl-hexyl phosphate, di-2-ethyl-hexyl phosphite, hydroxyethyl methacrylate monophosphate, glyceryl dimethacrylate phosphate, glyceryl-2-phosphate, glycerylphosphoric acid, methacryloxyethyl phosphate, pentaerythritol triacrylate monophosphate, pentaerythritol trimethacrylate monophosphate, dipentaerythritol pentaacrylate monophosphate, and dipentaerythritol pentamethacrylate monophosphate), toluene sulfonic acid, tribromoacetic acid, trifluoroacetic acid, trifluoromethanesulfonic acid, and trihydroxybenzoic acid. Mixtures of such acids can be used if desired. In one aspect, the organic acid is succinic acid or citric acid.

Since an organic, inorganic acid or mixture of both types of acids can be present, the solution is acidic. It has been found that it is best to have a pH of below about 4. However, a suitable pH range includes a range from about 2 to about 4.

The silane is present in the etch solution in an amount between about 20 and about 60 parts by weight. Suitable ranges of silane include from about 25 to about 50 parts by weight, about 30 to about 40 parts by weight, and about 20 to about 30 parts by weight, based on the total weight of the solution. The silane component of the composition can include more than one silane.

The silane compound, when at least partially hydrolyzed, contain at least one OH group and contain at least one non-hydrolyzable polymerizable organic group, both groups being bonded to a silicon atom. Upon hydrolysis, the silanol is monomeric, that is, it contains one silicon atom. Suitable polymerizable organic groups include vinyl, acryl, methacryl, glycidyl, allyl and styryl groups. In certain embodiment, the polymerizable groups are acryl or methacryl. Silanols have the formula RₙSi(OH)₄₋ₙ wherein R is a polymerizable organic group of the type described above and n is one to three, and in certain embodiments one.

It should be understood that the polymerizable group and the silicon atom can be attached via a “spacer” group. Generally, the spacer group is an alkyl chain (a C1 to about C10 carbon chain) that is attached to the polymerizable group via an oxygen bond and the silicon via an oxygen bond. These can be viewed as condensation products between a carboxylic acid, a silane having the formula RₙSi(X)₄₋ₙ, as described herein below and a diol having the C1 to C10 carbon chain. One hydroxyl group of the diol reacts with the carboxylic acid (or ester) of the polymerizable group to form an ester. The second hydroxyl of the diol group reacts with the hydrolyzable group of the silane to form a carbon-oxygen-silicon bond.

Hydrolyzable silanes have the formula RₙSi(X)₄₋ₙ wherein R and n are as defined above and X is a hydrolyzable group. Suitable hydrolyzable groups include alkoxy, substituted alkoxy, acyloxy, substituted acyloxy, and halogen. In one embodiment the hydrolyzable groups are alkoxy groups, such as methoxy and ethoxy groups. Generally, the organofunctional hydrolyzable silane is substantially free of amine groups, since silanols formed therefrom tend to polymerize spontaneously in water solution.

Suitable silanes that can be used to make the silanols useful in the etch solutions of the invention include acrylate silanes, methyacrylate silanes, mercapto silanes, and epoxy silanes. Commercially available hydrolyzable silanes include “A-151” vinyltriethoxysilane, “A-172” vinyltri(2-methoxyethoxy)silane, “A-174” gamma-methacryloxypropyltrimethoxysilane, “A-186” 3,4-epoxycyclohexylmethyltrimethoxysilane, “A-187” gamma-glycidoxypropyltrimethoxysilane, and “A-189” gamma-mercaptopropyltrimethoxysilane (all commercially available from Union Carbide Corp.), “Z-6030” gamma-methacryloxypropyltrimethoxysilane, “Z-6040” gamma-glycidoxypropyltrimethoxysilane, and “XZ-8-0951” gamma-mercactopropyltrimethoxysilane (all commercially available from Dow Corning Corp.), “A0564” allyltriethoxysilane, “D4050” diallyldichlorosilane, “D6205” divinyldiethoxysilane, “G6720” glycidoxypropyltriethoxysilane, “M8542” methacryloxypropyl-dimethylchlorosilane, and “S1588” m,p-styrylethyltrimethoxysilane (all commercially available from Petrarch Systems, Inc.), and mixtures thereof.

Optionally, the etch solutions of the invention can include a solvent. The terms “solvent” or “organic solvent” are recognized in the art and are intended to mean those components, other than water, that can be added to the etch solutions of the invention to help solubilize the components and aid in the evaporation of the etch solution from the substrate surface after application. Suitable solvents include those which are known to be pharmacologically acceptable for treatment of dental surfaces. These solvents include dimethyl sulfoxide, ethyl acetate, alcohols and ethers such as methanol, ethanol, propanol, butanol, ethylene glycol, propanediol, butanediol, pentanediol, butenediol, glycerin, trimethylolpropane, hexanetriol, allyl alcohol, diethylene glycol, diethylene glycol monomethyl ether, diethylene glycol monoethyl ether, triethylene glycol, triethylene glycol monomethyl ether, tetraethylene glycol, propylene glycol, dipropylene glycol, tripropylene glycol, 2-methoxyethanol, 2-ethoxyethanol, 2-(methoxyethoxy) ethanol, 2-isopropoxyethanol, 2-butoxyethanol, 1-methoxy-2-propanol, 1-ethoxy-2-propanol, dipropylene glycol, dipropylene glycol monomethyl ether, tripropylene glycol monomethyl ether, glycerine ether and the like, as well as ketones such as acetone, methyl ethyl ketone and the like and mixtures thereof. The solvent is present in an amount from about 1 to about 50 parts by weight, from about 5 to about 40 parts by weight or from about 10 to about 30 parts by weight.

Optionally, a surfactant can be included in the etch solution. The term “surfactant” is recognized in the relevant art to include those compounds which modify the nature of surfaces, e.g. reducing the surface tension of water. Surfactants are generally classified into four types: cationic (e.g. modified onium salts, where part of the molecule is hydrophilic and the other consists of straight or branches long hydrocarbon chains such as hexadecyltrimethyl bromide), anionic, also known as amphiphatic agents (e.g., alkyl or aryl or alkylarylsulfonates, carboxylates, phosphates), nonionic (e.g., polyethylene oxides, alcohols) and ampholytic or amphoteric (e.g. dodecyl-beta-alanine, such that the surfactant contains a zwitterionic group). One or more surfactants can be used in the present invention.

Cationic surfactants useful as surface tension reducing agents in the present invention include long chain hydrocarbons which contain quaternarized heteroatoms, such as nitrogen. Suitable cationic surfactants include quaternary ammonium compounds in which typically one of the groups linked to the nitrogen atom is a C12-C18 alkyl group and the other three groups are short chained alkyl groups.

Anionic surfactants (amphiphatic agents) are characterized by a single lipophilic chain and a polar head group which can include sulfate, sulfonate, phosphate, phosphonate and carboxylate. Exemplary compounds include linear sodium alkyl benzene sulfonate (LAS), linear alkyl sulfates and phosphates, such as sodium lauryl sulfate (SLS) and linear alkyl ethoxy sulfates. Additional examples of anionic surfactants include substituted ammonium (e.g., mono-, di-, and tri-ethanolammonium), alkali metal and alkaline earth metal salts of C6-C20 fatty acids and rosin acids, linear and branched alkyl benzene sulfonates, alkyl ether sulfates, alkane sulfonates, olefin sulfonates, hydroxyalkane sulfonates, fatty acid monoglyceride sulfates, alkyl glyceryl ether sulfates, acyl sarcosinates. acyl N-methyltaurides, and alkylaryl sulfonated surfactants, such as alkylbenezene sulfonates.

Nonionic surfactants do not dissociate but commonly derive their hydrophilic portion from polyhydroxy or polyalkyloxy structures. Suitable examples of polyhydroxy (polyhydric) compounds include ethylene glycol, butylene glycol,1,3-butylene glycol, propylene glycol, glycerine, 2-methyl-1,3-propane diol, glycerol, mannitol, corn syrup, beta-cyclodextrin, and amylodextrin. Suitable examples of polyalkyloxy compounds include diethylene glycol, dipropylene glycol, polyethylene glycols, polypropylene glycols and glycol derivatives.

Other suitable nonionic surfactants include other linear ethoxylated alcohols with an average length of 6 to 16 carbon atoms and averaging about 2 to 20 moles of ethylene oxide per mole of alcohol; linear and branched, primary and secondary ethoxylated, propoxylated alcohols with an average length of about 6 to 16 carbon atoms and averaging 0-10 moles of ethylene oxide and about 1 to 10 moles of propylene oxide per mole of alcohol; linear and branched alkylphenoxy (polyethoxy) alcohols, otherwise known as ethoxylated alkylphenols, with an average chain length of 8 to 16 carbon atoms and averaging 1.5 to 30 moles of ethylene oxide per mole of alcohol; and mixtures thereof.

Additionally, suitable nonionic surfactants include polyoxyethylene carboxylic acid esters, fatty acid glycerol esters, fatty acid and ethoxylated fatty acid alkanolamides. Block copolymers of propylene oxide and ethylene oxide, and block polymers of propylene oxide and ethylene oxide with propoxylated ethylene diamine are also included as acceptable nonionic surfactants. Semi-polar nonionic surfactants like amine oxides, phosphine oxides, sulfoxides, and their ethoxylated derivatives are included within the scope of the invention.

Suitable amphoteric and zwitterionic surfactants which contain an anionic water-solubilizing group, a cationic group and a hydrophobic organic group include amino carboxylic acids and their salts, amino dicarboxylic acids and their salts, alkylbetaines, alkyl aminopropylbetaines, sulfobetaines, alkyl imidazolinium derivatives, certain quaternary ammonium compounds, certain quaternary phosphonium compounds and certain tertiary sulfonium compounds

Examples of anionic, nonionic, cationic and amphoteric surfactants that are suitable for use in the present invention are described in Kirk-Othmer, Encyclopedia of Chemical Technology, Third Edition, Volume 22, pages 347-387, and McCutcheon's Detergents and Emulsifiers, North American Edition, 1983, both of which are incorporated herein by reference.

Typical concentration ranges of surfactant that are useful in the present etch solutions are from about 0.01 parts by weight to about 10 parts by weight, from about 0.1 parts by weight to about 5 parts by weight, and from about 0.5 parts by weight to about 2.5 parts by weight.

It should be understood that all values, including non-whole values (integers), inclusive, within the ranges of all concentrations (parts by weight) provided throughout this specification are considered individually. That is, it is within the discretion of the operator to choose any percentage within the ranges provided for any of the components as described herein. Therefore, the ranges provided are not limiting in terms of more narrow ranges and individual values that are encompassed by the parameters of the ranges identified.

The etch solutions of the present invention further include one or more ethylenically unsaturated functional monomers as detailed below. Suitable concentration ranges are from about 1 to about 30 percent, from about 1 to about 5 percent, from about 5 to about 20 percent, and from about 7.5 to about 15 percent. In particular, small amounts of such monomers facilitate in the handling and drying aspects of the etch solution.

The phrase “ethylenically unsaturated monomer” includes those reactive agents that include a double bond that can undergo polymerization with other monomers to form a polymeric matrix. The polymerization can be between like monomers or mixtures of monomers. Additionally, the monomers that are ethylenically unsaturated can further react with polyethylenically unsaturated functional crosslinking monomers to form crosslinked networks.

Suitable ethylenically unsaturated monomers, include, but are not limited to ethyleneglycol acrylate phosphate (and methacrylate), 2-hydroxyethylacrylate (HEA), 2-hydroxyethylmethacrylate (HEMA), 2- and 3-hydroxypropylacrylate and methacrylate, 1,3 and 2,3-dihydroxypropylacrylate and methacrylate, acrylic acid, methacrylic acid, 2-trimethylammonium ethylmethacrylic chloride, 2-acrylamido-2-methylpropane-sulfonic acid, acrylamide, methacrylamide, 2-hydroxyethylacrylamide and methacrylamide, N,N-bis(2-hydroxyethyl)acrylamide and methacrylamide, N-alkyl-N-hydroxyethyl acrylamides and methacrylamides, 2- and 3-hydroxypropylacrylamide and methacrylamide, methacrylamidopropyltrimethylammonium chloride, gylcerol monomethacrylate and monoacrylate, various reactive ethylenically unsaturated phosphates, such as HEMA phenylphosphate, methacryloyloxyethyldecahydrogen phosphate (MDP), ethylene glycol methacrylate phosphate (EGMP), ethylene glycol methacrylate maleate and mixtures thereof. It is considered that where an acrylate monomer is suitable the methacrylate analog will likewise be suitable.

The present invention also pertains to curable composites that include (a) at least one ethylenically unsaturated functional monomer, (b) at least one polyethylenically unsaturated functional crosslinking monomer, (c) (optional) a fluoride ion agent, (d) (optional) a solvent (e) (optional) water, with the solvent and/or water being present in an amount to equal a total of 100 parts by weight of all components.

The phrase “polyethylenically unsaturated functional crosslinking monomer” is recognized in the art and is intended to include those crosslinking agents that have two or more reactive double bonds present within the monomeric backbone. The degree of unsaturation provides the ability to polymerize with other crosslinking agent(s) as well as ethylenically unsaturated monomers to form a network of polymerized material.

Suitable crosslinking monomers include, for example, 2-hydroxypropyl-1,3-diacrylate and dimethacrylate, 3-hydroxypropyl-1,2-diacrylate and dimethylacrylate, triethylene glycol dimethacrylate (TEGDMA) and diacrylate, pentaerythritol diacrylate and dimethacrylate, polyethyleneglycol (400) diacrylate and dimethacrylate, glycerol dimethacrylate and diacrylate and pentaerylthritol trimethacrylate and triacrylate, triethylene glycol dimethacrylate and acrylate, the reaction product of pyromellitic dianhydride with glycerol dimethacrylate (PMGDM), addition product of 2-hydroxyethyl (meth)acrylate and pyromellitic dianhydride (PMDM), 2,2′-bis[4-(3-methacryloxy-2-hydroxy propoxy)-phenyl]-propane (bis-GMA) and mixtures thereof.

The term “fluoride agent” is recognized in the art and is intended to include those reagents that provide a fluoride ion. Suitable reagents include fluoride salts, such as sodium fluoride, potassium fluoride, ammonium fluoride, cetyl ammonium fluorides, such as cetyl trialkylammonium fluorides, and mixtures thereof.

The fluoride agent, optionally, is present in an amount from about 0.5 to about 5 parts by weight, from about 1 to about 3 parts by weight, and from about 1 to about 2 parts by weight.

The curable composition generally includes a photoinitiator system. The photoinitiator system includes a light-sensitive initiator and a polymerization accelerator. A suitable light-sensitive initiator is camphorquinone and a suitable polymerization accelerator is ethyl N,N-dimethyl-4-aminobenzoic acid or N,N-dimethylaminoethyl methacrylate.

Since most of the polymerization reactions which ethylenically unsaturated compounds undergo, particularly acrylate and methacrylate compounds, proceed by a free radical mechanism, a free radical initiator is generally included in the monomer system. Any free radical initiator which is substantially non-toxic in the amounts employed in the composition; which does not react adversely with either the polymeric matrix, once formed, and cures within a few minutes is acceptable. The free radical initiator may be of the chemical type (redox system) in which a peroxide initiator and a polymerization accelerator react at ambient temperatures to initiate the polymerization of the monomer system. Alternatively, a photoinitiator system is used in which light, such as ultraviolet light, but preferably the visible portion of the spectrum, is employed as the energy source to stimulate the free radical initiator.

Examples of such chemical initiators include hydroperoxides, peresters or peroxides such as benzoyl peroxide, or amines, tertiary aromatic amines, such as N,N-dimethyl toluidine can be used. Suitable photoinitiators include benzil 2,3-butanedione, phenyl-1,2-propandione, and camphorquinone (CQ).

Suitable light sources that are effective to cause photoinitiation to occur include halgoen, laser, plasma and LED sources. The amount of energy required, is that amount which is sufficient to initiate a photochemical reaction such that polymerization of the ethylenically unsaturated components is started or enhanced. One skilled in the art can determine the amount of light required to cause such reaction to occur and many light systems are commercially available.

The present invention further provides packaged formulations of the etch solution components, the curable compositions and combinations thereof.

For example, the present invention provides a packaged formulation having a container of aqueous acidic etching solution and a separate container of silane as described herein. Instructions are provided for combining the aqueous acidic solution and the silane and the application of the etching solution to a suitable substrate, i.e., a tooth, such that the substrate is conditioned for further treatment with a curable composition. The instructions provide the length of time to apply the solution, how to remove the excess solution (if necessary), and how to bond a restorative material, e.g., a fixture, to the treated substrate. Additionally, the packaged formulation can include a bonding resin composition as described herein or those that are commercially available.

The containers that can be used for the packaged products are those that are generally commercially available. Any container suitable for retaining the liquids can be used that does not react with the solution(s). The container can be a reusable bottle, as known in the art, a single use bottle or pouch. The container can be collapsible, such as those foil packets known in the art. In certain embodiments, it is advantageous to provide a container that does not permit light to penetrate through the bottle. The solutions can be stored at or below room temperature, so that the container should be to withstand temperatures below room temperature. Bottles or packets prepared from polyethylene or polypropylene are suitable as well as those aluminized foil packets prepared from various terephthalates.

The compositions of the invention are prepared by first combining acid, water, ethylenically unsaturated monomer and, optionally an additive such as a solvent. Next, the hydrolyzable silane is added with agitation for a period time e.g., a few seconds to a few minutes to an hour or more, sufficient to ensure that the silane becomes substantially hydrolyzed and/or miscible with the aqueous solution. If desired, additives such as those describe herein can also be added to compositions of the invention.

The present invention also provides methods to use the etch solutions, alone or in combination with the curable compositions of the invention. It should be understood that the etch solutions of the present invention can be used with those commercially available bonding resins. The method of the invention involves applying an effective amount of an etching solution to the substrate. The etch solution can be any of the solutions described herein, and for example, can include an inorganic acid, an organic acid, a solvent and water. The excess etching solution is removed from the substrate such that the substrate remains moist. This can be easily accomplished by touching an absorbent material to the surface and blotting the excess from the surface. Thereafter, a curable composite is applied to the moist substrate, i.e., a tooth. The curable composites of the packaged formulations and methods which follow can further include photoinitiator systems as described throughout the specification.

The compositions of the invention can be used in any dental application where silane priming solutions have hitherto been employed. Such solutions typically are used in the mouth, for example to prime dental materials such as dental porcelain (e.g., feldspathetic, nepheline syenite, alumina-base and synthetic porcelains), dental metals and alloys (e.g., metals or alloys of gold, silver, platinum, palladium, chromium, and nickel) and cured dental composites, restoratives and adhesives. In certain embodiments, application of the solution is used in the repair of crowns and bridgework, especially where bonds to dental porcelain, metals or alloys must be made. The compositions of the invention can also be used in conventional silane priming applications if desired (e.g., the priming of particulate or fibrous fillers for composites, such as fumed silica particles and “E-Glass” and “Fiberglas” fibers).

Following application of a composition of the invention to a desired substrate, the solvent and water are permitted to evaporate. Not to be limited by theory, the evaporation helps promote condensation of the silanol. The resulting primed substrate exhibits enhanced adhesion to polymerizable resins. The primed substrate can be coated with a layer or layers of polymerizable unfilled dental adhesive and overcoated with a layer or layers of filled dental composite or restorative, in order to alter the shape of the substrate (e.g., to restore the shape of the cusp of a crown). The primed substrate also can be coated with a layer or layers of unfilled dental adhesive and overcoated with a layer or layers of filled dental adhesive, then mated with a second primed, dental adhesive-coated substrate (e.g., to cement together the pieces of a broken crown or bridge). Standard analysis techniques can be employed to examine the hydrolytic stability and effectiveness of the priming treatment (e.g., water immersion or thermocycling of bonded samples, followed by tensile or shear bond strength measurements).

In addition to ability to treat a dental material, the present invention provides methods for treating, and ultimately adhering a material, to enamel, dentin, and/or a dental material. This is unique in that currently available methods do not lend themselves to treating two types of surfaces simultaneously. This is a great advantage where a tooth has been broken and a porcelain crown, for example has been broken adjacent to the broken tooth surface. Treatment with the etch solution of the invention can be used on both surfaces; each surface can then be individually treated with an appropriate material.

The phrase “effective amount of an etching solution” or “effective amount of a dental etching solution” is that amount required to modify the surface architecture of the substrate, such as bone substrate. Not to be limited by theory, it is believed that the etching solution removes proteins, lipids, and other foreign materials from the surface of the substrate and penetrates into the substrate. The surface and the penetrated substrate area are cleaned and modified such that they are receptive toward further modification, i.e., a curable composite where reactive functionality such as ethylenic bonds can attach to the modified material. Additionally, the hydrolyzed silanol reacts with the surface via one or more hydroxyl groups. The reactive monomer portion can then be reacted with a curable composite. A skilled artisan can readily determine the amount of etch solution to apply to the substrate, however, only enough of the solution is required so as to wet the surface and keep it moist as the surface is modified during rubbing.

The phrases “removing excess etching solution” or “removing excess dental etching solution” is intended to mean that a sufficient amount of the etching solution is removed from the substrate so that the surface remains wetted, i.e., moist. This can be accomplished by a variety of ways, including but not limited to, contacting the wet surface with an absorbent material, i.e., a cotton swab or tissue, passing a stream of air over the surface, and other methods known in the art. Ideally, enough etch solution should remain so that the surface of the substrate remains moist and does not become dry.

Up until the time of the present invention, when repairs of fractured ceramic restorations were attempted, it often became confusing to the operator as to the proper approach and order of the treatment of the tooth and then to the restoration, as they have traditionally required very different protocols. Typically, when ceramic restorations are partially fractured, dentin and enamel surfaces are often exposed. Up to the present invention, dentin and enamel surfaces have required a very different treatment than does the ceramic, and further, the materials used for the repair of the ceramic can have a detrimental effect on the repair of the adjacent tooth structure.

Prior to the present invention, it was recommended that application of silanes to ceramic surfaces be treated with a buffered hydrofluoric acid, in concentrations ranging from 4-9% and for periods of 1-9 minutes. Hydrofluoric acid can etch the ceramic, leaving a microporous surface that is retentive for resin materials. However, there are always safety concerns when strong acids such as hydrofluoric acid are used in the mouth. Therefore, the present invention provides the advantage when bonding to ceramics, hydrofluoric acid is not required.

The present invention also provides a novel method to bond resin to ceramic restorations without the need for hydrofluoric acid and to permit repairs to ceramics with simultaneous treatment of dentin and enamel surfaces adjacent to fractured ceramic restorations.

It has been discovered that the mixing if a drop of the SIMPLICITY 1® liquid (5% by weight nitric acid, 2.5% by weight succinic acid and 2.5% by weight methacrylic acid with the balance being water) and a drop of pure silane (3-(trimethoxysilyl) propylmethacrylate) causes the rapid hydrolysis and/or miscibility of the silane. The hydrolysis can be observed by watching the mixture as initially the SIMPLICITY 1® will lie on top of the silane similar to what one would see if one were to place a drop of oil on water. Over the course of about 30 seconds, the border of the drop of SIMPLICITY 1® expands in diameter until it is no longer visible as a separate liquid. The solution is then stirred for about 5 seconds and it is applied to a ceramic surface. It is allowed to dwell on the ceramic surface for about 30 seconds and then it is dried. A bonding resin, such as SIMPLICITY 2® (25% by weight PMGDM, 15% by weight HEMA, 8% by weight bis-GMA, 3% by weight ethyldimethylaminobenzoic acid (EDMAB) and 0.3% by weight camphoroquinone (CQ), with the balance being acetone, is then applied to the primed surface, dried and light-activated. Then the desired resin composite is applied, whether for purposes of bonding the ceramic restoration in place or for the purposes of repair. When this is repeated in the laboratory and a resin composite is applied and light-activated and shear testing performed, it resulted each and every time in the cohesive failure of the ceramic substrate, i.e. the bond between the ceramic primer and the ceramic is stronger than the cohesive strength of the ceramic tested (Vita Mark II ceramic). This has also been observed when testing a ceramic known as Procad. Procad and Vita Mark II are feldspathic ceramics and are very frequently used in dental ceramic restorations.

SIMPLICITY 2® is a resin adhesive system created for bonding resin composites to tooth structure. It has also been discovered that a mixture of the neat silane and SIMPLICITY 1® (as described above) can be used to bond tooth structure at the same time as it is used to bond to ceramics. The mixture is prepared as above, and is then applied to ceramic, dentin and/or enamel, allowed to dwell for 30 seconds and dried briefly. SIMPLICITY 2® is then applied and light activated for 10 seconds as is customary in the art. The desired restorative resin composite is then applied. The ability to treat ceramic, dentin and enamel surfaces at the same time is a novel and highly welcome development in restorative dentistry. It also permits the dentist to omit the potentially dangerous step of using hydrofluoric acid should the dentist choose to do so.

Historically, silane primers have not been thought to be stable long term, and it is commonly recommended that they be discarded after 12 months. It is not known for a fact whether this is truly accurate or whether it applies to all silane primers but it is certain that the ability to active immediate hydrolysis is a great advantage. Further, the ability to use the same mixture for the simultaneous treatment of enamel and dentin is a significant advantage.

Furthermore, it has been found that a solution, as described above, retains etch and bond properties for at least one month at 37° C. with strengths of between about 34 and about 40 mPa, where all cohesive failures within the ceramic and not the interface between the ceramic and tooth surface or ceramic/ceramic interface.

In another exemplary method, the substrate material can cleaned with isopropyl alcohol, rinsed with water and dried for at least 3 seconds. 1-2 drops of an aqueous acidic solution containing an ethylenically unsaturated monomer and silane are placed in an open well receptacle and agitated. A cotton or foam pellet is contacted to the solution. The moistened applicator is contacted to the substrate such that the area to be treated is wetted. The applicator is gently agitated with the solution against the surface for at least about 10 to about 30 seconds, i.e., from about 10 to about 30 seconds, from about 10 to about 20 seconds, or from about 10 to about 15 seconds.

Generally, the etch solution is allowed to remain on the substrate surface for approximately 10 to about 30 seconds, i.e., from about 10 to about 20 seconds, or from about 10 to about 15 seconds. Ideally, the preparation should not be allowed to dry. There is no requirement to rinse the etch solution from the surface before any further applications are done.

After the etching treatment is complete, from about 1 to about 3 coats of an adhesive resin, such as those that are available commercially or those described within the present application, can be applied directly over the moist conditioned preparation with a suitable applicator, such as a brush. The adhesive resin is dried gently, for about 5-8 seconds to facilitate evaporation of the solvent. After the surface is dried, the surface should appear to be “shiny,” if not, additional resin adhesive should be applied. The adhesive resin is then light-cured for about 10 seconds. Optionally, a restorative material can be applied to the adhesively treated surface to continue the procedure. However, depending upon what therapeutic treatment is intended, the adhesive resin can serve as a protectant covering and no further restorative treatment may be necessary.

For example, the etching/adhesive system can be used to seal enamel/dentin prior to restoration with light-cured or self-cured composite materials. Indirect Restorations are also encompassed by the present invention and include those preparations when using a light-cured, self-cured or dual-cured composite cement or glass ionomer or resin-modified glass ionomer cement. The methods and compositions of the invention can also be useful for desensitization to treat hypersensitive and/or exposed root surfaces. Additionally, the methods and compositions of the invention can be used to bond in a post and core.

Restorative materials applicable for use with the present invention include those known in the art. Composite materials, synthetic bone materials, bone-like apatite and hydroxyapatite materials are well suited for use with the compositions and methods of the invention. Suitable examples of dental restoratives include composite filling materials, inlays, onlays, crown, bridges, ceramics, veneers and Maryland bridges.

The invention is further illustrated by the following examples which in no way should be construed as being further limiting. The contents of all references, pending patent applications and published patent applications, cited throughout this application, including those referenced in the background section, are hereby incorporated by reference. It should be understood that the models used throughout the examples are accepted models and that the demonstration of efficacy in these models is predictive of efficacy in humans.

In the following examples, a suitable substrate was used as

identified below. The substrate, mounted in methylmethacrylate, was treated with a silane reagent as described below for 30 seconds, dried briefly if the resin adhesive was applied subsequently and dried thoroughly for 5 seconds if no adhesive was applied, optionally treated with an adhesive resin as described below, subjected to light activation for 10 seconds with a Demetron 401 light generating unit (Demetron-Kerr) and then a resin composite cylinder was adhered to the treated surface followed by light activation for an additional 40 seconds. Testing was performed on the resulting composite after the time noted below.

The samples were then subjected to shear bond strength testing on a Universal testing machine with a crosshead speed of 1 mm/min. The force required to break the composite was recorded in kg and converted to mPa on the basis of the surface area of the sample.

All samples throughout the experiments were prepared prior to testing. The test sample was steam sterilized for 24 hours prior to treatment. For examples where a tooth (dentin or enamel) was studied, the tooth was stabilized and embedded into a cylindrical mounting with a polymerizable monomer, such as methyl methacrylate. The tooth surface was ground until an appropriate surface was exposed, i.e., enamel or dentin. The surface was finely polished, rinsed, dried and then treated. All samples were prepared this way; both with the etch and curable composites of the invention as well as those of competitive products. This was done for consistency between samples.

### EXAMPLES

### Example 1

A brushful of Ultradent silane (Ultradent, Utah) was applied to a ceramic composite (Vita Mark II ceramic, Vident Corporation) and allowed to dwell on the surface for 30 seconds, dried for 5 seconds, and then a cylinder of Flow-it ALC (referred to as FALC) (Pentron) was applied to the treated surface, via the mold, light activated for 40 seconds and tested for adhesive strength. Immediate bond strength was 6.3 mPa. Adhesive failure.

### Example 2

A brushful of Ultradent silane was applied to a ceramic composite (Vita Mark II ceramic, Vident Corporation) and allowed to dwell on the surface for 30 seconds, dried for 5 seconds and treated with a few drops of SIMPLICITY 2® resin, then subjected to light activation for 10 seconds with a Demetron 401 light generating unit (Demetron-Kerr). A cylinder of FALC was applied to the treated surface followed by light activation for an additional 40 seconds and tested for adhesive strength. Immediate bond strength was 15.2 mPa. Adhesive failure.

### Example 3

Several coats of SIMPLICITY 2® resin were applied to a ceramic composite (Vita Mark II ceramic, Vident Corporation) and subjected to light activation for 40 seconds with a Demetron 401 light generating unit (Demetron-Kerr). A cylinder of FALC was applied to the treated surface followed by light activation for an additional 40 seconds and tested for adhesive strength. Immediate bond strength was 13.4 mPa. Adhesive failure.

### Example 4

A drop of SIMPLICITY 1® and a drop of 3-(trimethoxysilyl)propylmethacrylate were placed in a mixing well together for about 30 seconds until the silane became miscible/hydrolyzed in the aqueous solution. The resultant solution was stirred for about 5 seconds and then applied to a ceramic composite (Vita Mark II ceramic, Vident Corporation) for 30 seconds. The surface was dried for 5 seconds, several coats of SIMPLICITY 2® resin were applied to the dried surface and subjected to light activation for 10 seconds with a Demetron 401 light generating unit (Demetron-Kerr). A cylinder of FALC was applied to the treated surface followed by light activation for an additional 40 seconds and tested for adhesive strength. Immediate bond strength was 20.6 mPa. Cohesive failure.

### Example 5

Several coats of SIMPLICITY 2® resin were applied to a ceramic composite (Vita Mark II ceramic, Vident Corporation) and subjected to light activation for 10 seconds with a Demetron 401 light generating unit (Demetron-Kerr). A cylinder of FALC was applied to the treated surface followed by light activation for an additional 40 seconds and tested for adhesive strength. Bond strength after 24 hours (stored in water at 37° C.) was 13.3 mPa. Adhesive failure.

### Example 6

A brushful of Ultradent silane (Ultradent, Utah) was applied to a ceramic composite (Vita Mark II ceramic, Vident Corporation) and allowed to dwell on the surface for 30 seconds and dried for 5 seconds. A cylinder of FALC was applied to the treated surface and tested for adhesive strength. Bond strength after 24 hours (stored in water at 37° C.) was 6.4 mPa. Adhesive failure.

### Example 7

A brushful of Ultradent silane (Ultradent, Utah) was applied to a ceramic composite (Vita Mark II ceramic, Vident Corporation) and allowed to dwell on the surface for 30 seconds, and dried for 5 seconds. Several coats of SIMPLICITY 2® resin were applied to the dried surface and subjected to light activation for 10 seconds with a Demetron 401 light generating unit (Demetron-Kerr). A cylinder of FALC was applied to the treated surface followed by light activation for an additional 40 seconds and tested for adhesive strength. Bond strength after 24 hours (stored in water at 37° C.) was 34.4 mPa. Cohesive failure.

### Example 8

A drop of SIMPLICITY 1® and a drop of 3-(trimethoxysilyl) propylmethacrylate were placed in a mixing well together for about 30 seconds until the silane became miscible/hydrolyzed in the aqueous solution. The resultant solution was stirred for about 5 seconds and then applied to a ceramic composite (Vita Mark II ceramic, Vident Corporation) for 30 seconds. The surface was dried for 5 seconds, several coats of SIMPLICITY 2® resin were applied to the dried surface and subjected to light activation for 10 seconds with a Demetron 401 light generating unit (Demetron-Kerr). A piece of FALC was applied to the treated surface followed by light activation for an additional 40 seconds and tested for adhesive strength. Bond strength after 24 hours (stored in water at 37° C.) was 34.4 mPa. Cohesive failure.

### Example 9

A few drops of Ultradent HF gel (9.4% hydrofluoric acid buffered gel, Ultradent, Utah) were applied to a ceramic composite (Vita Mark II ceramic, Vident Corporation) and allowed to dwell on the surface for 2 minutes as recommended by the manufacturer. The treated surface was rinsed with water and dried for 5 seconds. Several coats of SIMPLICITY 2® resin were applied to the dried surface and subjected to light activation for 10 seconds with a Demetron 401 light generating unit (Demetron-Kerr). A cylinder of FALC was applied to the treated surface followed by light activation for an additional 40 seconds and tested for adhesive strength. Bond strength after 24 hours (stored in water at 37° C.) was 34.3 mPa. Cohesive failure.

### Example 10

Ultradent HF gel (9.4% hydrofluoric acid buffered gel, Ultradent, Utah) was applied to a ceramic composite (Vita Mark II ceramic, Vident Corporation) and allowed to dwell on the surface for 2 minutes as recommended by the manufacturer. The treated surface was rinsed with water, dried for 5 seconds and a few drops of Ultradent silane (Ultradent, Utah) were applied to the treated surface and allowed to dwell on the surface for 30 seconds, then dried for 5 seconds. Several coats of SIMPLICITY 2® resin were applied to the dried surface, dried for 5 seconds and subjected to light activation for 10 seconds with a Demetron 401 light generating unit (Demetron-Kerr). A cylinder of FALC was applied to the treated surface followed by light activation for an additional 40 seconds and tested for adhesive strength. Bond strength after 24 hours (stored in water at 37° C.) was 31.2 mPa. Cohesive failure.

### Example 11

A few drops of RelyX Ceramic Primer (item number 2721) (Minnesota Manufacturing & Mining, St. Paul, Minn.) were applied to a ceramic composite (Vita Mark II ceramic, Vident Corporation) and allowed to dwell on the surface for 30 seconds and dried for 5 seconds. A cylinder of FALC was applied to the treated surface followed by light activation for an additional 40 seconds and tested for adhesive strength. Bond strength after 24 hours (stored in water at 37° C.) was 33.7 mPa. Adhesive failure.

### Example 12

A drop of SIMPLICITY 1® and a drop of 3-(trimethoxysilyl) propylmethacrylate were placed in a mixing well together for about 30 seconds until the silane became miscible/hydrolyzed in the aqueous solution. The resultant solution was stirred for about 5 seconds and then applied to a ceramic composite (Vita Mark II ceramic, Vident Corporation) for 30 seconds. The surface was dried for 5 seconds, several coats of SIMPLICITY 2® resin were applied to the dried surface and subjected to light activation for 10 seconds with a Demetron 401 light generating unit (Demetron-Kerr). A cylinder of FALC was applied to the treated surface followed by light activation for an additional 40 seconds and tested for adhesive strength. The sample was stored in water at 37° C. for 14 days (stored in water at 37° C.) to examine bond stability over time. Bond strength after 14 days was 34 mPa. Cohesive failure.

### Example 13

A few drops of RelyX Ceramic Primer (item number 2721)(Minnesota Manufacturing & Mining, St. Paul, Minn.) were applied to dentin (prepared as described above) and allowed to dwell on the surface for 30 seconds and dried for 5 seconds. Several coats of SIMPLICITY 2® resin were applied to the dried surface and subjected to light activation for 10 seconds with a Demetron 401 light generating unit (Demetron-Kerr). A cylinder of Z-100 composite was applied to the treated surface followed by light activation for an additional 40 seconds and tested for adhesive strength. Immediate bond strength was 14.8 mPa. Adhesive failure.

### Example 14

A drop of SIMPLICITY 1® and a drop of 3-(trimethoxysilyl) propylmethacrylate were placed in a mixing well together for about 30 seconds until the silane became miscible/hydrolyzed in the aqueous solution. The resultant solution was stirred for about 5 seconds and then applied to dentin (prepared as described above) and allowed to dwell on the surface for 30 seconds. The surface was not dried but several coats of SIMPLICITY 2® resin were applied to the surface, dried for 5 seconds, two additional coats of SIMPLICITY 2® resin were applied to the treated surface and dried again. The coated surface was subjected to light activation for 10 seconds with a Demetron 401 light generating unit (Demetron-Kerr), a cylinder of Z-100 composite was applied to the treated surface followed by light activation for an additional 40 seconds and tested for adhesive strength. Immediate bond strength was 30.7 mPa. Adhesive failure. Bond strength after 24 hours (stored in water at 37° C.) was 43.4 mPa. Adhesive failure.

### Example 15

A drop of SIMPLICITY 1® and a drop of 3-(trimethoxysilyl) propylmethacrylate were placed in a mixing well together for about 30 seconds until the silane became miscible/hydrolyzed in the aqueous solution. The resultant solution was stirred for about 5 seconds and then applied to enamel (prepared as described above) and allowed to dwell on the surface for 30 seconds. The surface was not dried but several coats of SIMPLICITY 2® resin were applied to the surface, dried for 5 seconds, two additional coats of SIMPLICITY 2® resin were applied to the treated surface and dried again. The coated surface was subjected to light activation for 10 seconds with a Demetron 401 light generating unit (Demetron-Kerr), a cylinder of Z-100 composite was applied to the treated surface followed by light activation for an additional 40 seconds and tested for adhesive strength. Immediate bond strength was 23.6 mPa. Adhesive failure. Bond strength after 24 hours (stored in water at 37° C.) was 40.7 mPa. Adhesive failure.

### Example 16

Several coats of SIMPLICITY 2® resin were applied to dentin (prepared as described above) and subjected to light activation for 10 seconds with a Demetron 401 light generating unit (Demetron-Kerr). A cylinder of Z-100 composite was applied to the treated surface followed by light activation for an additional 40 seconds and tested for adhesive strength. Immediate bond strength was 28.5 mPa. Adhesive failure. Bond strength after 24 hours (stored in water at 37° C.) was 43 mPa. Adhesive failure.

### Example 17

Several coats of SIMPLICITY 2® resin were applied to enamel (prepared as described above) and subjected to light activation for 10 seconds with a Demetron 401 light generating unit (Demetron-Kerr). A cylinder of Z-100 composite was applied to the treated surface followed by light activation for an additional 40 seconds and tested for adhesive strength. Immediate bond strength was 24.8 mPa. Adhesive failure. Bond strength after 24 hours (stored in water at 37° C.) was 43 mPa. Adhesive failure.

Cohesive failure denotes that the breakage occurred in the ceramic composite material or tooth, not at the bond interface between the tooth surface and ceramic composite. Adhesive failure denotes that the bond interface between the tooth surface and the ceramic composite underwent failure.

Although the present invention has been described with reference to preferred embodiments, persons skilled in the art will recognize that changes may be made in form and detail without departing from the spirit and scope of the invention. All references cited throughout the specification, including those in the background, are incorporated herein in their entirety. Those skilled in the art will recognize, or be able to ascertain, using no more than routine experimentation, many equivalents to specific embodiments of the invention described specifically herein. Such equivalents are intended to be encompassed in the scope of the following claims.

I claim:

1. A dental solution comprising
an aqueous acid solution;
a silane; and
an ethylenically unsaturated monomer, wherein the pH of the dental solution is below about 4.

2. The dental solution of claim 1, wherein the silane is present in an amount between about 20 and about 60 percent by weight of the total weight of the solution.

3. The dental solution of claim 2, wherein the silane is selected from one or more of an acrylate silane, a methyacrylate silane, a mercapto silane, and an epoxy silane.

4. The dental solution of claim 3, wherein the silane is partially or fully hydrolyzed into a silanol.

5. The dental solution of claim 1, wherein the acid is an inorganic acid.

6. The dental solution of claim 5, wherein the inorganic acid is selected from the group consisting of hydrochloric, nitric, sulfuric and phosphoric acids.

7. The dental solution of claim 6, wherein the inorganic acid is present in an amount between about 1 and about 10 percent by weight of the total weight of the solution.

8. The dental solution of claim 7, wherein the inorganic acid is nitric acid.

9. The dental solution of claim 8, wherein the nitric acid is present in an amount between about 3 and about 7 percent by weight of the total weight of the solution.

10. The dental solution of claim 1, further comprising an organic acid.

11. The dental solution of claim 10, wherein the organic acid is present in an amount between about 1 and about 5 percent by weight of the total weight of the solution.

12. The dental solution of claim 11, wherein the organic acid is succinic acid.

13. The dental solution of claim 12, wherein the succinic acid is present in an amount between about 2 and about 3 percent by weight of the total weight of the solution.

14. The dental solution of claim 1, wherein the ethylenically unsaturated monomer contains a carboxylic acid moiety.

15. The dental solution of claim 1, wherein the ethylenically unsaturated monomer is present in an amount between about 1 and about 5 percent by weight of the total weight of the solution.

16. The dental solution of claim 15, wherein the ethylenically unsaturated monomer is acrylic or methacrylic acid.

17. The dental solution of claim 16, wherein the acrylic acid or methacrylic acid is present in an amount between about 2 and about 3 percent by weight of the total weight of the solution.

18. The dental solution of claim 1, further comprising a solvent.

19. The dental solution of claim 11, wherein the solvent is an alcohol or a ketone.

20. The dental solution of any of claim 1, wherein the silane is trimethoxysilylpropyl methacrylate at a concentration of about 50 percent of the total weight of the dental solution, the ethylenically unsaturated monomer is methacrylic acid and is present at a concentration of about 2.5 percent of the total weight ot the dental solution, the pH of the dental solution is about 2, and the solution further comprises nitric acid at a concentration of about 5 percent of the total weight of the dental solution and succinic acid at a concentration of about 2.5 percent of the total weight of the dental solution.

21. A method to treat a dental material, comprising the steps of:
applying to the dental material a dental solution comprising the dental solution as claimed in claim 1; and
allowing the dental material to remain moist.

22. The method of claim 21, wherein the dental material comprises feldspathetic, nepheline synenite, alumina-base, dentin, enamel, or synthetic dental porcelain.

23. The method of claim 21, wherein the dental material comprises dental metal or alloy comprising gold, silver, platinum, palladium, chromium, or nickel.

24. The method of claim 21, wherein the dental material comprises a crown or bridge.
